(19) United States
(12) Patent Application Publication
(10) Pub. No.: US 2014/0336533 A1
(43) Pub. Date: Nov. 13, 2014

(54) NON-INVASIVE DEVICE FOR REMOVING EXUDATE FROM A WOUND, USE THEREOF AND KIT COMPRISING SAID DEVICE

(71) Applicants: Laboratoires Urgo, Chenove (FR); Universite Paul Sabatier Toulouse III, Toulouse (FR); INSERM (Institut National de la Sante et de la Recherche Medicale), Paris (FR)

(72) Inventors: Christophe Dardenne, Fontenilles (FR); Bernard Pipy, Toulouse (FR); Michel Lamoise, Bessey Les Citeaux (FR)

(21) Appl. No.: 14/366,347

(22) PCT Filed: Dec. 21, 2012
(86) PCT No.: PCT/FR2012/053068

§ 371 Date: Jun. 18, 2014

(30) Foreign Application Priority Data
Dec. 22, 2011 (FR) 1162295

(51) Int. Cl. A61B 10/00

(52) U.S. Cl. CPC A61B 10/0045
USPC 600/573

(57) ABSTRACT

The invention relates to a non-invasive device (1) that can be used to remove exudate from a wound, comprising a chamber (2) having a side wall (3), an open lower end (4) and an upper end (5), an access opening being disposed at the upper end (5) and/or in the side wall (3) of the chamber (2). The device (1) is intended to be placed in contact with the skin around the wound during use by means of an interface (7). The invention also relates to the uses of the device and to a kit comprising same.

### FIELD OF THE APPLICATION

The present invention relates to a medical device which makes it possible to easily collect the exudates from a wound in order to analyze said exudates in real time, thus enabling cellular, biochemical and molecular monitoring of the progression of the wound healing, and which also makes it possible to visually observe the development of the healing of a wound.

### PRIOR ART

The healing of a wound is a natural physiopathological process, human and animal tissues being capable of repairing localized lesions by means of repair and regeneration processes which are characteristic features of said tissues.

The rapidity and the quality of the healing of a wound depend on the general condition of the affected organism, on the etiology of the wound, on the condition and the location of the wound, and on the possible occurrence of an infection, and also on genetic factors which possibly predispose to healing disorders.

The natural healing of a wound occurs mainly according to three successive phases, each of these phases being characterized by specific cell activities which make the repair process progress according to precise chronological sequences: the inflammatory phase, the granulation phase (or proliferative phase), and the maturation phase, giving the definitive appearance of the scar.

The first phase, the inflammatory phase, begins as soon as there is rupturing of the blood vessels, which triggers the formation of a clot (blood coagulation) mainly composed of fibrin and fibronectin, and which will constitute a provisional matrix. This matrix partly fills the lesion and will enable the migration within the damaged area of inflammatory cells recruited to perform detersion of the wound. The platelets present will also release factors (for example cytokines, growth factors) enabling the recruitment of cells involved in the healing process. This phase is characterized by the infiltration and activation, on the site of the lesion, of numerous inflammatory cells (such as neutrophil polymorphonuclear cells), but also cells which provide cleaning of the wound or detersion (such as macrophages).

The second phase corresponds to the development of the granulation tissue. Colonization of the wound through fibroblast proliferation is first observed. Then, the migration of endothelial cells from the healthy vessels will enable neovascularization, or angiogenesis, of the damaged tissue. In the granulation tissue, the fibroblasts are activated and will differentiate into myofibroblasts which have considerable contractile properties, generated by the actin microfilaments, enabling wound contraction. These microfilaments are expressed through a protein: smooth muscle α-actin. These myofibroblasts therefore play a major role in the formation and maturation of the granulation tissue which will result in healing of the lesion. Keratinocyte migration and reconstruction of the epidermis then take place.

This phase is initiated by a decrease in the inflammatory state of the lesion, accompanied by apoptosis of the inflammatory cells.

The third phase of the process is mainly a step of maturation aimed at reconstructing a functional tissue which is as identical as possible to the tissue of origin. The granulation tissue previously formed therefore undergoes remodeling. Part of the extracellular matrix is digested by proteases (essentially matrix metalloproteases and elastases), and gradual reorganization of the extracellular matrix is observed. Gradually, type III collagen, which is predominant in the granulation tissue, is replaced with type I collagen, the main matrix component of the dermis. At the end of the maturation phase, the fibroblasts, myofibroblasts and vascular cells experience a reduction in their proliferation and/or their activity. The excess cells then die through apoptosis. In parallel to the remodeling of the extracellular matrix and to the apoptosis of the excess cells.

The inflammatory phase is an essential phase of healing and should be transient. In some cases, it can be prolonged by the presence of certain infectious agents such as Staphylococcus aureus or Pseudomonas aeruginosa or by a preexisting pathological condition such as diabetes, an immune deficiency or else venous insufficiency. The resolution of the inflammation is a critical point which conditions the priming of tissue repair. Disruption of this phase will cause an abnormal extension of the inflammatory phase and will result in lesion chronicity by delaying tissue repair. The resolution of inflammation is a dynamic phenomenon which involves apoptosis of the inflammatory cells, elimination thereof and the appearance of anti-inflammatory, chemotactic and angiogenic mediators. One way to evaluate the correct development of this critical period, namely the resolution phase, is to analyze the exudates from the wound (cells and mediators). Indeed, inflammatory chronicity is reflected by a greater inflow of inflammatory cells, and an excessive production of numerous inflammatory cytokines and chemokines and of proteases which degrade the cellular matrix. Conversely, the resolution phase involves the appearance of macrophages of a certain type, responsible for phagocytosis of the apoptotic inflammatory cells. These macrophages are also involved in the production of mediators that promote the granulation phase and also tissue repair. Analysis of the exudate will enable the physician to evaluate the inflammatory phase and the resolution thereof, and will thus make it possible to choose to treat the wound with an appropriate therapeutic agent so as to promote better healing.

Usually, wounds are covered with a dressing or a compress in order to prevent contamination by infectious agents present in the environment, but also in order to keep the wound in a moist environment promoting healing. However, this protection system does not make it possible to analyze exudates or to visualize healing in real time. Furthermore, the exudate accumulates in the dressing and saturates the absorbent medium of the dressing. The dressing must therefore be changed regularly in order to avoid maceration of the wound, which involves a risk of contamination of the wound since it is momentarily exposed to the open air. Furthermore, dressings can also adhere, even weakly, to the wound, and thus cause lesions to the wound when they are removed. These further attacks cause the inflammation to continue, resulting in an extension of the inflammatory phase and therefore a substantial modification of the healing kinetics, something which the Applicant's device remedies effectively.

Implantable chambers have been developed for studying dermal wound healing on animals such as mice or pigs. However, these implantable chambers are partly inserted under the animal's skin and are kept in place by sutures. This method is therefore invasive, i.e. it has the major drawback of requiring a surgical procedure and therefore risks causing an inflammatory reaction in the subject in question, by creating attacks on the tissues when it is implanted. These attacks on the tissues will have harmful consequences on the correct development of healing. Furthermore, these implantable chambers have essentially been developed for modeling the mechanisms of healing in animals.

Finally, the company Acueity, in its patent WO 2004/045674, describes a device for collecting the exudate from mammary glands. The device in question comprises a receptacle containing an absorbent pad which is in contact with the nipple and the device is kept in place over the nipple by means of an adhesive. The exudate is absorbed by the pad and cannot therefore be analyzed in real time; furthermore, the exudates harvested in the pad are not pure and risks of loss and/or of degradation at the time they are extracted from the pad can be noted. This device does not have an opening system and must therefore be removed in order to recover the exudates from the pad for analytical purposes.

There is therefore a real need for a non-invasive, leaktight and conformable device which makes it possible to directly and passively collect pure exudates for the purposes of real-time analysis (in particular in order to control the development of the inflammatory phase and the initiation of the granulation phase) and which makes it possible to visualize the progression of the healing without necessarily having to open said device, or to remove it, thus ensuring protection of the internal environment of the device, and therefore protection of the wound, with respect to the external environment (i.e. with respect to external infectious agents, but also to the animal itself, or to its fellow creatures, in other words with respect to scratching, biting, soiling, impacts or else rubbing) and with respect to the device itself.

### INVENTION

A first object of the present invention is a non-invasive device which makes it possible to collect the exudates from a wound, comprising a chamber having a side wall, an open lower end and an upper end, there being an access opening on the upper end and/or on the side wall of said chamber, said device being intended to be in contact with the skin and to surround the wound during use by means of an interface.

According to a preferred embodiment, said device comprises a removable means for closing the chamber, intended to be placed in the closed position, on the access opening of the chamber.

According to one particular embodiment of the device, the interface is an adhesive placed on the lower end of the chamber of said device.

During use, the device according to the invention is simply stuck onto the healthy skin so as to surround the patient's wound without coming into contact with said wound, by virtue of its adhesive interface. During healing, the wound weeps and the exudate flows into the device. Once a sufficient amount of exudate for analyses has been collected in the device, the exudate is sampled, for example by opening the closing means. It should be noted, furthermore, that, when the exudate is sampled regularly, the wound does not macerate, thereby promoting rapid healing.

The device according to the invention may in particular be considered to be a passive device for harvesting exudates, as opposed to an active device. The term “active device” is intended to mean a device wherein the exudates are harvested through the application of an external effect or stress, which may be physical and/or mechanical, such as a negative pressure, for instance vacuum devices or negative pressure therapy devices, also known as NPT devices. The active and passive devices can also differ from one another by virtue of the quality of the exudates harvested. Indeed, when a mechanical and/or physical stress is introduced into the active device, the exudates harvested are of a larger volume, and there is thus a dilution of the various cells, mediators or cell factors contained in said exudates. Furthermore, with the active devices, the cells found in the exudates may be dead and/or activated. The term “activated cells” is intended to mean cells extracted from their natural environment which are in a situation of stress and which may then lose, inter alia, their functional capacities, which is, for example, the case with macrophages and neutrophil polymorphonuclear cells. The advantage of the device of the present invention is therefore to make it possible to preserve the integrity and the functionalities of the various cell populations contained in the exudates from the wound. A true “image” of the wound is thus obtained.

Furthermore, the device of the present invention is a device for direct harvesting of exudates on the healing site. The term “device for direct harvesting” is intended to mean a device in which the exudates are directly sampled at their source of emission, in the present case in the vicinity of the wound, the exudate does therefore not need to transit through a related chamber or receptacle and/or through an absorbent material before its extraction from the device. The direct harvesting thus makes it possible to preserve the integrity and the physiology of the wound and of its environment, all without disrupting the wound healing. Thus, the device according to the invention is centered on the macroscopic and microscopic analysis of the exudates from the wound, but also of the wound itself.

Said device according to the invention is devoid of any absorbent means coming into contact with the exudate and the wound. This makes it possible to avoid maceration of the wound due to the saturation of said absorbent means by the exudate, as may be the case with a dressing. Furthermore, the absence of absorbent means makes it possible to avoid any trapping or degradation of proteins, metabolites or cells present in the exudate, all with the objective of recovering pure exudates without the loss of cells and metabolites.

According to a particular embodiment, the device is leaktight in the interaction zone device/subject's skin. Thus, the exudate collected in the chamber cannot escape via the edges of said device. It is also possible to add a lip made of conformable, semi-rigid, optionally shape-memory material, such as, in particular, PMMA (poly(methyl methacrylate)), POM-C (polyacetal copolymer), PEKK (polyether ketone ketone), silicone, polyurethane or else polyamide, on the lower part of the holding element, which lip may prove to be useful when the surface on which the device is attached is not flat, thus making it possible to ensure the leaktightness of said device even further. According to a particular embodiment, the device comprises a means for carrying out gas exchanges between the inside of said device and the outside air in order to ensure good wound healing by preventing hypoxia and/or anoxia. Said means for carrying out gas exchanges is preferentially a filter which makes it possible to keep the inside of the device sterile, i.e. to prevent contamination by the outside environment. Examples of filters that can be used on the device according to the invention may in particular be filters made of polyurethane, nonwovens, frits (porous disks of compressed silica) or else a sterile membrane. The filter may in particular be placed on, under or in the closing means, but also on, under or in the side walls of the chamber, the external part of said filter still being in contact with the environment external to the device.

According to a particular embodiment, the device comprises a graduation system for evaluating the amount of exudate contained in the device.

The chamber of the device may in particular be a tubular-shaped component, preferentially made of rigid material such as PMMA (poly(methyl methacrylate)), POM-C (polyacetal copolymer), PEKK (polyether ketone ketone) or polyamide. Optionally, the chamber of the device may be made of flexible material, such as, in particular, polyurethane or silicone.

The tubular-shaped chamber must have an internal volume that is sufficiently high to collect all the exudate generated by a wound, i.e. 5000 g/m²/24 h. However, in order not to bother the patient too much, the height of the device should preferentially be less than 1 centimeter. In order to characterize this chamber more clearly, it is possible to describe it in terms of its maximum exudate capacity which is less than 1 ml/cm², preferentially less than 700 μl/cm², and even more preferably less than 500 μl/cm².

Advantageously, said device also comprises an optionally tubular-shaped holding element, which is circumscribed to or inscribed in said chamber, and which is open at both its ends. The expression “holding element which is circumscribed to the chamber” is intended to mean, for the purpose of the present invention, a holding element which surrounds, on the outside, the side wall of the chamber in its lower part. The expression “holding element which is inscribed in the chamber” is intended to mean, for the purpose of the present invention, a holding element which is placed on the inside of the chamber, in the vicinity of the side wall and in the lower part of the chamber.

According to a preferred embodiment, the tubular-shaped holding element is made of a shape-memory semi-rigid material such as, in particular, PMMA (poly(methyl methacrylate)), POM-C (polyacetal copolymer), PEKK (polyether ketone ketone), silicone, polyurethane or polyamide.

According to another preferred embodiment, the holding element and the tubular chamber each have a means for making them rigidly interconnected. This means may be a screw thread, a clip, a pin, a bayonet system, a locking system and/or optionally adhesive.

When the device has a holding element which is circumscribed to the chamber, the cross section of the tubular chamber is smaller than the cross section of the holding element. When the device has a holding element which is inscribed in the chamber, the cross section of the tubular chamber is larger than the cross section of the holding element. In either of these embodiments, it is important for the smallest opening of the lower end of the device to have a surface area larger than that of the wound to be treated, so as to surround it. In the case where the holding element is inscribed in the tubular chamber, the cross section of said chamber may narrow at its upper part, such as a bottleneck. In another embodiment, the upper part of said chamber may have a diameter larger than the holding element, or else the same diameter, in which case the wound will be completely visualized.

Numerous shapes can be envisioned, both for the cross section of the tubular chamber and for that of the holding element, such as, in particular, circular, oval, elliptical, square or rectangular. According to a preferred embodiment, the chamber and the holding element have the same shape. However, it is not necessary for the chamber and the holding element to have the same shape. It is possible, for example, to envision fitting a cylindrical tubular chamber to a parallelepipedal holding element.

According to a preferred embodiment, the lower end of the tubular chamber and/or of the holding element may in particular have an external conformable flange made of flexible material, such as, for example, polyurethane or silicone.

According to another particular embodiment, said flange may be an element dissociated from the chamber or from the holding element. It is characterized as a perforated element made of flexible material, said flexible material being chosen from the list of flexible materials as previously defined, and surrounds the external part of the holding element/tubular chamber assembly, as proximally as possible with respect to this chamber.

When the device does not comprise a holding element, the interface of said device is an adhesive placed on the lower end of the chamber, and in particular on the flange if the latter is present. The presence of the flange makes it possible to increase the contact area between the adhesive interface of the device and the patient's skin, thus improving the holding of the device in place on the skin.

When the device comprises a holding element, the interface is an adhesive placed on a skirt made of flexible material. Said skirt made of flexible material is held between the side wall of the chamber and the holding element with a means for making these three elements rigidly interconnected, such as a screw thread, a clip, a pin, a locking system and/or optionally adhesive. The flexible material of the skirt may in particular be chosen from the group comprising conformable flexible supports such as films, foams, silicones and nonwovens. This flexibility makes it possible to fit the device to non-planar areas such as, in particular, a phalanx, a knee or an elbow. Thus, the term “skirt” is intended to mean, for the purpose of the present invention, an element comprising a single perforation, which may in particular widen and be frustrum-shaped. The interface may in particular be planar when it is not integrated into the device and may have a widened shape when it is attached between the tubular chamber and the holding element.

The leaktightness of said device is preferentially provided by the juxtaposition of the tubular chamber and of the holding element, between which constituents the skirt made of flexible material is inserted. Said skirt in addition confers good-quality conformability properties to the entire device. The term “good-quality conformability” is intended to mean that the skirt made of flexible material adapts to the surface to which it is attached, by taking an appropriate shape. The device, once attached to a planar surface or a non-planar surface, makes it possible to retain the entire volume of the exudates harvested at the level of the wound. The same is true for the volume of a solution which is injected into said device through a septum found on the chamber or on the closing means. The leaktightness of said device is calculated by means of a relatively simple measuring method which is easy to implement and which is described in example 4.

This method is implemented both on a planar surface and on a non-planar surface. The implementation of this method on a non-planar surface makes it possible to measure the leaktightness of said device which is the object of the invention in situations where the conformability of the latter appears to be essential.

It should be noted that, whatever the type of surface, for example a planar or non-planar surface, to which the device is attached, the leaktightness of said device is demonstrated. Thus, the device which is the object of the present invention combines leaktightness and conformability by means of the juxtaposition of the tubular chamber, of the holding element and of the skirt made of flexible material.

The interface should not come into contact with the patient's wound. It is attached on the perimeter of the edges of the wound on the patient's healthy skin, all with the aim of surrounding the wound. The interface must be sufficiently strong for the device to be able to stay in place on the skin for several days without the leaktightness, at the level of the skin/device zone, of the device being affected.

The skirt may have various types of possible shapes, such as simple square, round, rectangular or triangular shapes, or shapes with rounded edges, or more sophisticated shapes in the shape of stars, or else in the shape of a cloverleaf, the branches of which may or may not be of the same dimensions, in order to be able to fit to the place on the body on which the device is attached. For the cloverleaf shape, this has the advantage of not allowing creases in its proximal parts of the chamber, and also enables improved conformability according to the position occupied on the part of the subject's body on which the device is placed.

According to a preferred embodiment, the free surface of the interface is covered with a protective barrier such as, in particular, a plastic film. This protective barrier preserves the adhesive properties of the device and prevents any contamination by dust or by microorganisms before the use of said device.

It is also possible to envision that the adhesive constituting said interface is pre-coated, i.e. the adhesive has been applied during the manufacture of the device or else coated by the user at the time of use of the device, i.e. once the device has been removed from its protective wrapper. The coating may in particular be carried out by means of a brush, or else of a spatula, i.e. by manual coating, before placing said device on the subject's skin.

The adhesive, constituting the interface, may in particular be chosen from the group comprising surgical adhesives, filmogels, nitrocellulose, collodion, cyanoacrylate, hydrocolloids, acrylic adhesives, UV adhesives or else hot-melt adhesives.

The removable means for closing said device makes it possible to easily gain access to the wound. Said removable closing means fits to the access opening(s) of the chamber, preferentially to the upper end of the chamber, by means of a screw thread, a clip, a pin, a crimping means and/or a bayonet system. Preferably, said closing means fitted to the chamber in the closed position, by means for example of a screw thread, makes it possible to keep the environment closed. The junction between said closing means and the chamber, provided by any means mentioned above, for instance by means of a screw thread, is therefore leaktight.

This closing means may preferentially be made of semi-rigid or rigid material such as PMMA (poly(methyl methacrylate)), POM-C (polyacetal copolymer), PEKK (polyether ketone ketone), polyamide, silicone, or else of any metal material, such as aluminum.

According to a particular embodiment, said removable closing means is connected to the tubular chamber by a linking means, such as in particular a plastic linker. This makes it possible to avoid losing said closing means when the latter is separated from the tubular chamber. According to a particular embodiment, the closing means can easily be removed in order to photograph or film the wound and the development thereof.

According to a preferred embodiment, the device comprises a means for visualizing the wound. Thus, the tubular chamber and the closing means may be made of transparent materials. It is also possible to envision a transparent window, optionally equipped with a magnifying lens, on the means for closing the device. The advantage of this visualizing means is that the development of the healing or of any infection that might occur can be monitored. In addition, this monitoring is carried out without direct intervention on the wound, which makes it possible to avoid any incorrect manipulation of the wound or the re-opening thereof. Furthermore, this makes it possible to visualize the wound without the latter being exposed to the external environment that may contain pathogenic agents, guaranteeing non-contamination of the interior of the tubular chamber. The closing means may also have a diaphragm. When the diaphragm is in the open position, the user can see the wound and when the diaphragm is in the closed position, the wound is no longer visible.

It is important to note that the device which is the object of the present invention is a device, the final purpose of which is strictly speaking not to treat the wound. It is preferably a means for both macroscopically and microscopically monitoring the wound, the exudate and its cellular and molecular environment, as described in the patent application filed by Laboratoires Urgo under number FR 11 62344.

According to a preferred embodiment, the device comprises at least one septum or a microvalve intended for sampling or injecting a liquid. This septum makes it possible in particular to sample the exudate without removing the device, which preserves the sterility of the internal environment. This septum may also be used for injecting a solution in order to clean or disinfect the wound. The septum may also be used for injecting a solution containing a pharmacological agent or a therapeutic agent, such as, in particular, an antibiotic, a painkiller, an anti-infective, or else a healing agent. Due to the absence of an absorbent means in the device, the injected solution will be in direct contact with the wound, contrary to a dressing. The septum may be placed on the closing means or on the wall of the tubular chamber and, optionally, of the holding element. Advantageously, two tubes comprising closing means, such as septa or microvalves, can be positioned on either side of the side wall of the tubular chamber in order to facilitate washing of the wound. Thus, the cleaning solution, for example, is injected via a septum, circulates in the device, and is removed via the second septum.

The exudate may also be collected from the device, for analytical purposes, by opening the closing means or directly by sampling “in situ”. The collected exudate contains all of the factors and metabolites produced during healing. The exudate also contains live and functional cells which can optionally be put back into culture in order to carry out phenotypic and functional studies, ex vivo. Furthermore, the exudate may contain pathogenic agents, such as bacteria, which can easily be identified after sampling, thereby enabling the physician to prescribe an appropriate treatment.

One or more embodiments of the device according to the invention may also be combined. Thus, for example, the invention may relate to a device which comprises at least one septum or one microvalve, intended for sampling or injecting liquid and/or a means capable of carrying out gas exchanges between the interior of said device and the exterior.

The invention will be understood more clearly on reading the following figures which are merely illustrations and could not in any way limit the invention.

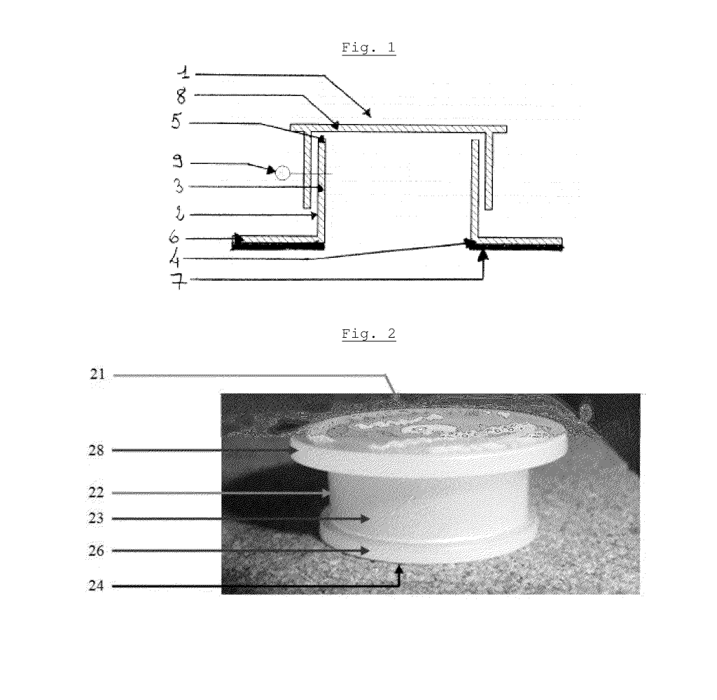

FIG. 1 is a transverse sectional view of one of the embodiments of the invention. The device (1) comprises a tubular chamber (2) comprising a side wall (3), and two open ends, which are a lower end (4) and an upper end (5). Said lower end (4) of the tubular chamber (2) has an external flange (6) made of flexible material. The interface (7) of the device (1) comprises of a thin layer of surgical adhesive, applied to the lower surface of the flange (6) of the tubular chamber (2). A means (8) for closing the device (1) is attached to the upper part of the tubular chamber (2) by means of a pin (9).

FIG. 2 represents a photograph of an embodiment of a device (21) comprising a tubular chamber (22) having a side wall (23) and two open ends, which are a lower end (24) and an upper end. The lower end (24) has an external flange (26). A closing means (28) is attached to the upper part of the tubular chamber (22) by means of a clip (not represented here).

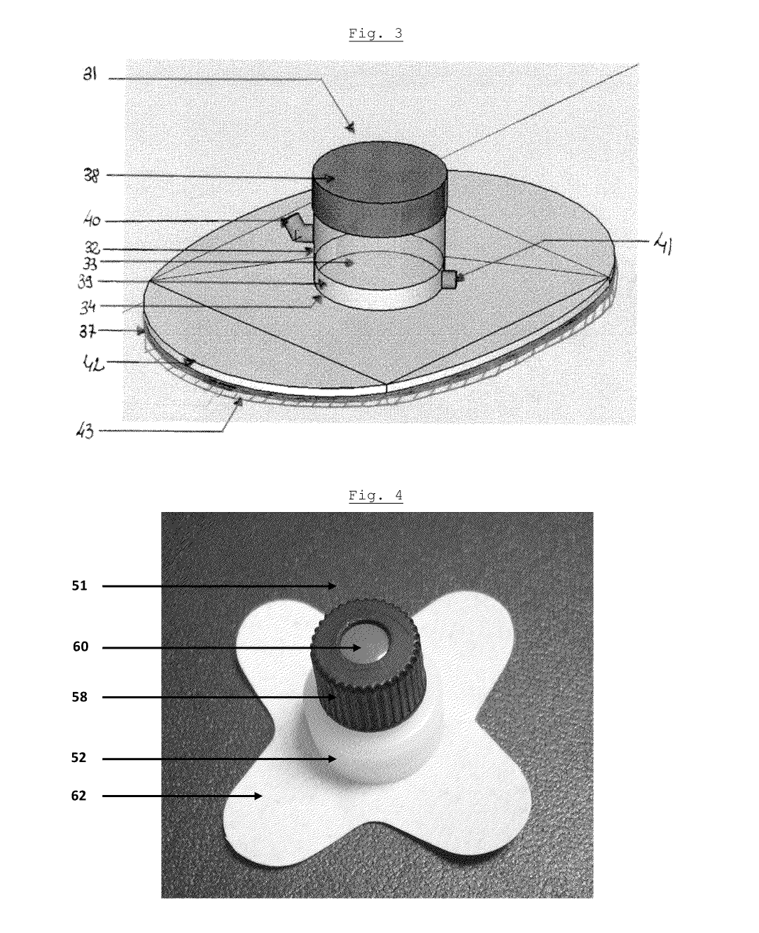

FIG. 3 is an exploded diagrammatic representation of a device (31) according to the invention comprising a cylindrically shaped tubular chamber (32) and of a holding element (39) having a cross section with a diameter smaller than the diameter of the tubular chamber (32). Said holding element (39) is thus inscribed in the lower end (34) of said tubular chamber (32) and is held by a clip. The side wall (33) of the tubular chamber (32) has two diametrically opposed septa (40 and 41) which make it possible to easily wash the wound. Since the second septum (41) is located at the level of the holding element (39), the latter therefore has an opening that has approximately the same size as that of the septum (41). The interface (37) comprises an adhesive placed on the lower surface of a skirt (42) made of flexible material, which is in the form of an element comprising a single perforation, which widens, in the shape of a frustrum. Said skirt is attached between the holding element (39) and the side wall (33) of the tubular chamber (32), which makes it possible to hold the skirt in place. The free surface of the adhesive interface (37) is protected by a protective film (43). A closing means (38) is attached to the upper part of the tubular chamber (32) by means of a screw thread (not visible here).

FIG. 4 represents a photograph of another embodiment of said device according to the invention. This device (51) is made up of a tubular chamber (52) and a holding element which is inscribed in the tubular chamber (52) and is attached by means of a clip (not visible here). The interface comprises an adhesive placed on the lower surface of a skirt (62) made of flexible material which is held in place between the holding element and the side wall of the tubular chamber (52). The free surface of the interface is protected by a protective film (not visible here). A closing means (58) is fitted to the upper part of the tubular chamber (52) by means of a screw thread. Said closing means (58) has, at its center, a septum (60) which is used to sample the exudate inside the device (51) without having to open the device.

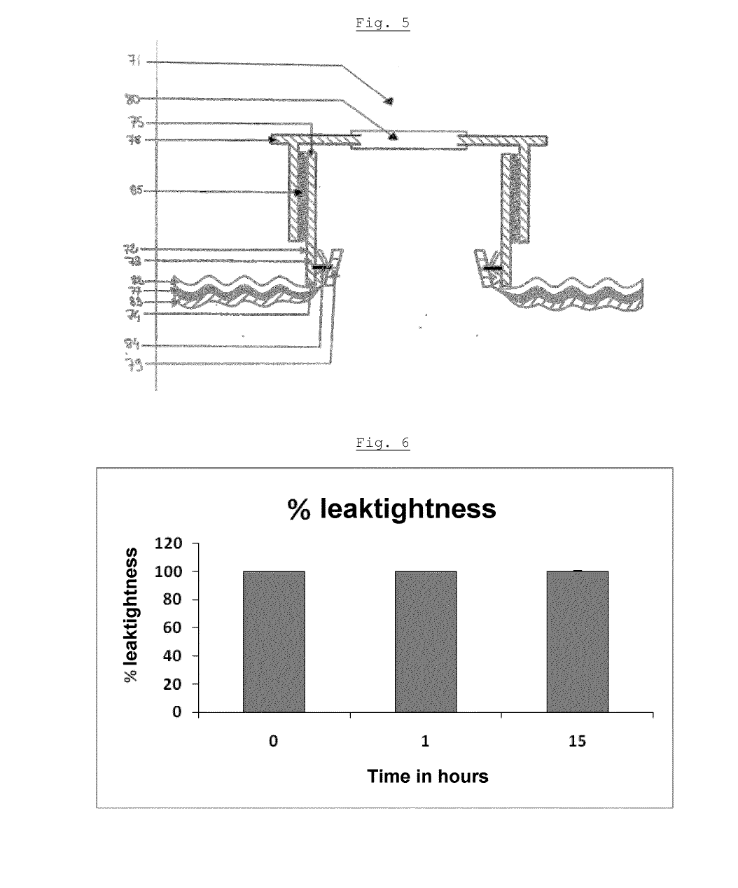

FIG. 5 is a transverse sectional view of a device (71) according to the invention, comprising a tubular chamber (72) comprising a side wall (73), and two open ends, which are a lower end (74) and an upper end (75). A holding element (79) is inscribed in the lower end (74) of said tubular chamber (72) and is held in place by a clip (84). The interface (77) of the device comprises an adhesive placed on the lower surface of a skirt (82) made of flexible material, corresponding to a frustrum-shaped tubular element, i.e. its upper opening has a smaller diameter than its lower opening. Said upper opening of the skirt is attached between the holding element (39) and the side wall (33) of the tubular chamber (32) by a clip (84), which makes it possible to hold the skirt in place. The free lower surface of the adhesive interface (77) is protected by a protective film (83). A means (78) for closing the device (71) is attached to the tubular chamber by means of a screw thread (85). Said closing means is made of transparent material in order to visualize the wound and it has at its center a septum (80) in order to collect the exudate.

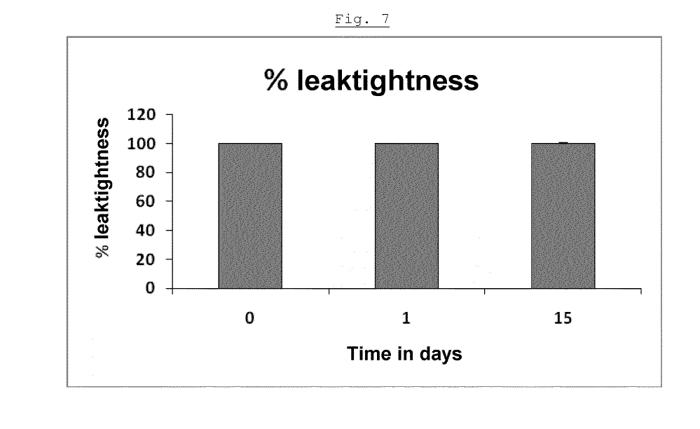

FIG. 6 is a curve representing the leaktightness percentage of the device produced according to example 1, said device being attached to a planar surface, as a function of time.

FIG. 7 is a curve representing the leaktightness percentage of the device produced according to example 1, said device being attached to a non-planar surface, as a function of time.

Another object of the invention is the use of the previously described device for following and/or monitoring the development of the healing of a wound. Indeed, the device makes it possible to collect and analyze the exudate from a wound. In order to follow the healing development of a wound, the device should be placed over the wound. It is then necessary to wait for a sufficient amount of exudate to accumulate in the device. In order to optimize the recovery of the factors and mediators produced and also of the cells which may adhere to the wound, washing of the wound using a physiological solution may be envisioned. The exudate contained in the device may then be sampled by opening the device or with a syringe through the septum. The exudate thus sampled can be analyzed in order to determine the state of progression of the inflammatory phase and of the healing in general or to know whether an infectious agent is present in the wound.

According to one particular mode of use of said device, a solution is introduced into the device after the latter has been applied to the skin. This solution may contain a disinfecting and/or cleaning agent which makes it possible to make the wound and the internal environment of the device clean and sterile. This solution may also contain a therapeutic agent or a pharmacological agent, such as, in particular, an antibiotic, a painkiller, an anti-infective or else a healing agent.

According to one particular mode of use of said device, the exudates sampled from this device will enable real-time analysis thereof.

In one particular mode of use of said device, the exudate is sampled through a septum or a microvalve, present on the device. This sampling makes it possible to collect the exudate without removing the device and without direct intervention on the wound.

Finally, according to another particular mode of use of said device, the latter may make it possible to wash the wound.

Another object of the invention relates to a kit for collecting exudate from wounds, comprising:
at least one device as previously described;
at least one recovering device, such as a micropump or a syringe;
optionally a needle.

According to a preferred embodiment, the kit comprises at least one solution to be injected into said device. The solution to be injected comprises a disinfecting and/or cleaning active ingredient and/or a therapeutic agent. This solution makes it possible to sterilize the environment of the wound and/or to treat an infection and/or the wound.

The invention will be described in greater detail by means of the following examples which are given purely by way of nonlimiting illustration.

### EXAMPLES

### Example 1

### Device for Collecting the Exudate from a Wound

The device is such as that of FIG. 5, and comprises:
a chamber (72) with a circular cross section, 0.5 cm in height and 1 cm in diameter, made of poly(methyl methacrylate);
a holding element (79) with a circular cross section, 0.2 cm in height and 0.9 cm in diameter, made of polyether ketone ketone, which is inscribed in the chamber (79);
an interface (77) comprising an acrylic adhesive precoated onto the lower surface of a skirt (82) made of a flexible film;
a clip (84) for attaching the holding element (79) and the skirt (82) made of a flexible film to the tubular chamber (72);
a protective plastic film (83) protecting the free surface of the adhesive interface;
a closing means (78) with a circular cross section, made of transparent polyurethane, in order to be able to visualize the wound, which is attached to the tubular chamber (72) by a screw thread (85) and which comprises a septum (80) intended for sampling the exudate.

### Example 2

### Use of the Device on a 0.75 cm² Wound

It is desired to follow the healing of a wound of approximately 0.75 cm² present on the left forearm of a patient.

For this, the device of example 1 is used.

The closing system (78) of the device is opened in order to visualize the bottom part of the tubular chamber (72) and of the holding element (79). The protective film (83) is removed from the adhesive interface (77). The adhesive interface (77) is placed on the left forearm of the patient around the latter's wound, taking care to ensure that the interface (77), the tubular chamber (72) and the holding element (79) do not come into contact with the wound. A light pressure is exerted for 2 to 3 minutes in order to ensure that the device is properly held on the forearm. The closing means (78) is screwed onto the chamber. At a predetermined time, a washing solution is injected via the septum (80) of the means (78) for closing the tubular chamber (72), and then the exudate is sampled via the septum by means of a syringe fitted with a needle. The exudate is analyzed in order to verify that no infectious agent is present in the wound.

### Example 3

### Kit

The kit contains the device according to example 1 and also two syringes, two needles, a washing solution and a sterile bottle. The sterile device is put in place as in example 2 and sterilized by means of the washing solution which is injected through the septum using a syringe fitted with a needle according to example 2. The second syringe and the second needle make it possible to evacuate the disinfecting solution after washing of the wound. The exudate sampled is transferred into the sterile bottle for the purpose of biological tests.

### Example 4

### Measurement of the Leaktightness of the Device of Example 1 Attached to a Planar or Non-Planar Surface

In order to measure the leaktightness of the device produced according to example 1, said device is attached to a planar or non-planar glass surface. In the present case, the non-planar glass surface is the side surface of a 250 ml volumetric flask.

A volume of 500 μl of an aqueous solution, such as water, is then added into said device. The latter is closed using the closing means. As soon as the device has been closed (time 0 h also subsequently referred to as T0), the assembly: glass support, closed device, water is weighed.

This assembly is again weighed 1 hour after (time 1 h also subsequently referred to as T1) and 15 hours after (time 15 h also subsequently referred to as T15) the closing of the device.

The test is carried out three times for the planar surface and three times for the non-planar surface (number 1, 2 and 3).

In order to determine the leaktightness percentage (subsequently referred to as %) at a given time, the following calculation is carried out:

% leaktightness=(weight of the assembly at time T)/(initial weight of the assembly at T0).

The mean of the weight over the three tests is calculated at T0, T1 and T15 in the following way:

Mean(weight)=[weight test 1+weight test 2+weight test 3]/3.

The mean of the leaktightness percentage over the three tests is calculated at T1 and T15 in the following way:

Mean(%)=[% test 1+% test 2+% test 3]/3.

The results obtained for the planar surface are given in the following table and the change in the leaktightness percentage as a function of time is represented in FIG. 6:

PLANAR SURFACE 1 h

| | Weight 0 h | Weight 1 h | % |
|---|---|---|---|
| Number 1 | 67.17 | 67.17 | 100 |
| Number 2 | 61.29 | 61.28 | 99.9836841 |
| Number 3 | 60.57 | 60.57 | 100 |
| Mean | 63.01 | 63.0066667 | 99.9945614 |
| SEM | | | 0.00543863 |

PLANAR SURFACE 15 h

| | Weight 0 h | Weight 15 h | % |
|---|---|---|---|
| Number 1 | 67.17 | 67.163 | 99.9895787 |
| Number 2 | 61.29 | 60.978 | 99.4909447 |
| Number 3 | 60.57 | 60.56 | 99.98234902 |
| Mean | 63.01 | 62.9003333 | 99.8213378 |
| SEM | | | 0.16520593 |

The device produced according to example 1 is therefore leaktight on a planar surface. Indeed, the difference between the leaktightness percentage at T+1 hour and the leaktightness percentage at T+15 hours is not significant.

The results obtained for the non-planar surface are given in the following table and the change in the leaktightness percentage as a function of time is represented in FIG. 7:

NON-PLANAR SURFACE 1 h

| | Weight 0 h | Weight 1 h | % |
|---|---|---|---|
| Number 1 | 101.8 | 101.8 | 100 |
| Number 2 | 103.41 | 103.38 | 99.9709893 |
| Number 3 | 99.74 | 99.74 | 100 |
| Mean | 101.65 | 101.64 | 99.9903298 |
| SEM | | | 0.00967024 |

NON-PLANAR SURFACE 15 h

| | Weight 0 h | Weight 15 h | % |
|---|---|---|---|
| Number 1 | 101.8 | 101.62 | 99.8231827 |
| Number 2 | 103.41 | 103.36 | 99.9516488 |
| Number 3 | 99.74 | 99.74 | 100 |
| Mean | 101.65 | 101.57 | 99.9249438 |
| SEM | | | 0.05276032 |

The device produced according to example 1 is therefore leaktight on a non-planar surface in addition to being conformable. Indeed, the difference between the leaktightness percentage at T+1 hour and the leaktightness percentage at T+15 hours is not significant.

The device produced according to example 1 therefore combines leaktightness and conformability thanks to the juxtaposition of the tubular chamber, of the holding element and of the skirt made of flexible material.

1. A non-invasive device (1, 21, 31, 51, 71) which makes it possible to collect the exudates from a wound, comprising a chamber (2, 22, 32, 52, 72) having a side wall (3, 23, 33, 73), an open lower end (4, 24, 34, 74) and an upper end (5, 75), there being an access opening on the upper end (5, 75) and/or on the side wall (3, 23, 33, 73) of said chamber (2, 22, 32, 52, 72), said device (1, 21, 31, 51, 71) being intended to be in contact with the skin and to surround the wound during use by means of an interface (7, 37, 77).

2. The device (1, 21, 31, 51, 71) according to claim 1, wherein it comprises a removable means (8, 28, 38, 58, 78) for closing the chamber (2, 22, 32, 52, 72), intended to be placed in the closed position, on the access opening of the chamber (2, 22, 32, 52, 72).

3. The device (1, 21, 31, 51, 71) according to claim 1, wherein the interface (7, 37, 77) is an adhesive placed on the lower end (4, 24, 34, 74) of the chamber (2, 22, 32, 52, 72).

4. The device (31, 51, 71) according to claim 1, wherein it also comprises a holding element (39, 79), optionally of tubular shape, which is circumscribed to or inscribed in said chamber (32, 52, 72), and open at both its ends.

5. The device (31, 51, 71) according to claim 4, wherein the interface (37, 77) is an adhesive placed on a skirt (42, 62, 82) made of flexible material, said skirt (42, 62, 82) being held between the side wall (33, 73) of the chamber (32, 52, 72) and the holding element (39, 79).

6. The device (31, 51, 71) according to claim 5, wherein said flexible material of the skirt (42, 62, 82) is chosen from the group comprising conformable flexible supports such as films, foams, silicones and nonwovens.

7. The device (1, 21, 31, 51, 71) according to claim 1, wherein the adhesive constituting said interface (7, 37, 77) is precoated or coated at the time of use.

8. The device (31, 51, 71) according to claim 4, wherein the holding element (39, 79) and the tubular chamber (32, 52, 72) each have a means for making them rigidly interconnected.

9. The device (1, 21, 31, 51, 71) according to claim 1, wherein the lower end (4, 24, 34, 74) of the chamber (2, 22, 32, 52, 72) and/or of the holding element (39, 79) comprises a conformable flange (6, 26) made of flexible material.

10. The device (31, 51, 71) according to claim 3, wherein said holding element (39, 79) is made of shape-memory semi-rigid material.

11. The device (1, 21, 31, 51, 71) according to claim 1, wherein said removable closing means (8, 28, 38, 58, 78) fits to the access opening(s) of the chamber (2, 22, 32, 52, 72) by means of a screw thread (85), a clip, a pin (9), a crimping means and/or a bayonet system.

12. The device (1, 21, 31, 51, 71) according to claim 1, wherein it comprises at least one septum (40, 41, 60, 80), or one microvalve, intended for sampling or injecting liquid, and/or a means capable of carrying out gas exchanges between the interior of said device (1, 21, 31, 51, 71) and the exterior.

13. The device (1, 21, 31, 51, 71) according to claim 1, wherein it comprises a means for visualizing the wound.

14. The use of the device (1, 21, 31, 51, 71) as defined in claim 1, for monitoring the healing of a wound, and/or for introducing a solution into the device and/or for analyzing the exudates from the wound in real time.

15. A kit for collecting the exudate from wounds, comprising:
at least one device (1, 21, 31, 51, 71) as defined in claim 1;
at least one recovering device, such as a micropump or a syringe;
optionally a needle.
